(19) United States
(12) Patent Application Publication
(10) Pub. No.: US 2010/0274226 A1
(43) Pub. Date: Oct. 28, 2010

(54) Deployment Catheter

(75) Inventor: Palle M. Hansen, Bjaeverskov (DK)

Correspondence Address: BRINKS HOFER GILSON & LIONE/CHICAGO/COOK, PO BOX 10395, CHICAGO, IL, 60610, US

(73) Assignees: Cook Incorporated, Bloomington, IN (US); William Cook Europe ApS, Bjaeverskov (DK)

(21) Appl. No.: 12/676,471

(22) PCT Filed: Sep. 5, 2008
(86) PCT No.: PCT/US08/75458

§ 371 Date: May 20, 2010

Related U.S. Application Data
(60) Provisional application No. 60/967,639, filed on Sep. 6, 2007.

(51) Int. Cl. A61M 25/00

(52) U.S. Cl. 604/523

(57) ABSTRACT

A catheter for use in an endoluminal delivery assembly includes a device holding region or element on which there is provided a plurality of flexible fingers extending outwardly therefrom. The fingers act to maintain a device to be deployed in the correct position on the catheter and act to resist deformation of the device during deployment.

### FIELD OF THE INVENTION

The present invention relates to an introducer or deployment assembly for deploying implants and other prostheses within a patient, and in particular to the catheter or cannula which carries the implant or other prosthesis.

### BACKGROUND OF THE INVENTION

A typical endoluminal introducer or deployment system includes an inner catheter or cannula, which may also be arranged as a pusher and/or dilator (hereinafter referred to as an inner catheter or catheter element) and a sheath covering the inner catheter. An implant or prosthesis is carried on the inner catheter and is fixed thereto by means of the covering sheath and with or without one or more restraining wires or any of a number of other known retention systems.

The implant or prosthesis might be a stent, a stent graft, a vena cava filter, an occlusion device or any other implantable device of such a nature.

Once the distal end of the catheter has been positioned inside a patient, typically at the site of the patient's vasculature to be treated, the device is released and deployed in the desired position. The deployment operation involves retracting the covering sheath so as to expose the device to be implanted, which device is then deployed, either by self-expansion or by means of an expansion device such as an inflatable balloon. In the case where the device is also held by restraining wires, these are withdrawn, typically after retraction of the sheath. Restraining wires may or may not be used in such apparatus, generally in dependence upon the nature of the device to be deployed, size restrictions and the particular medical application or intervention procedure.

The step of retracting the covering sheath from the inner catheter has been known to compress or otherwise deform the device to be implanted. This can affect the positioning of the device at the deployment site and can in some circumstances damage the device itself. These problems can be experienced particularly in the case of delicate implants such as some stents.

Various systems have been proposed to deal with this problem. For example, US Patent Publication No. 2004/0106977 discloses in some embodiments the provision of one or more bands of an adhesive on the outer surface of the inner catheter, which is intended to hold a stent until its deployment, and in other embodiments ridges or stepped walls on the outer surface of the inner catheter which engage struts of the stent to prevent longitudinal movement thereof along the inner catheter as the covering sheath is retracted.

A problem with providing adhesive on the inner catheter is that this is another material to which a patient is exposed, even if only temporarily. It also requires a constant compressive force on the device held on the inner catheter for the glue to perform its function fully. The pressure required to compress the stent reliably into the adhesive layer results in there being a higher friction between the sheath and the stent, which provides an undesirable compromise in such devices.

The mechanical holding function provided by ridges or stepped walls on the inner catheter can be significantly better at holding the device firmly on the inner catheter during the deployment operation. However, there are risks that the ridges on the outer surface of the inner catheter can snag on the device once this has been deployed or on the inner surfaces of the patient's vasculature as it is retracted from within the patient. This can cause movement or damage to the implanted device or irritation or damage to the patient's vasculature or organs. The risks are increased where the device to be implanted is small and/or particularly delicate and when the device is implanted in or near a tortuous part of a patient's vasculature.

### SUMMARY OF THE INVENTION

The present invention seeks to provide an improved deployment assembly and an improved inner catheter or cannula.

According to an aspect of the present invention, there is provided a catheter element for an introducer designed to carry a medical device to be implanted in a patient, which medical device includes a structure with one or more interstices therein; the catheter element including an elongate device support region on which a device can be located and at least one flexible member arranged on at least a portion of said support region, said flexible member extending radially outwardly from said support region and being partially deformable by a said medical device carried on the catheter element so as to be at least partially locatable in the interstice or interstices of the medical device.

The device support element is typically a portion of the catheter designed to hold the device to be implanted.

The flexible fingers, which are preferably of a fibrous or filamentary type, are able to engage with the device being carried so as to provide support to the device in the longitudinal direction of the catheter, particularly upon the removal of a covering sheath. The flexible nature of the fingers prevents or substantially eliminates the risk of damage to the device or to the patient during withdrawal of the catheter once the device has been deployed. In particular, even with fingers which are substantially evenly flexible throughout their length, the tips of the fingers will be able to deflect more than their bases, with the result that if they come into contact with the deployed device or the walls of the patient's vasculature or organ, they will brush against these without causing damage.

It is to be understood that the term catheter element as used herein is intended to encompass all forms of device for carrying such implants and prostheses endoluminally in a patient, including inner catheters, cannulae and devices acting as pushers and/or dilators.

The fingers may be substantially flat, they may be substantially round or oval in cross-section or may have any other suitable cross-sectional shape.

In some embodiments, the fingers extend substantially perpendicularly to the longitudinal axis of the device support region. In other embodiments, the fingers extend at an angle to the transverse direction. A preferred embodiment has fingers which extend towards a distal end of the catheter, for example at 45° or at any angle between 20° to 80°, more preferably 30° to 60°.

It is envisaged that there could be a variety of different sets of fingers, arranged at different angles to one another.

Preferably, the fingers extend in a radial direction of the device support region.

The fingers may be substantially straight but they could be curved. Again, the catheter could be provided with a mixture or straight curved fingers.

In some embodiments, at least some of the fingers have hooked ends.

Preferably, the fingers are formed from the same material as the elongate element. This allows the fingers to be moulded with the device support region.

In another embodiment, the fingers are formed from a material different from the device support region and the catheter.

The length of the fingers will be dependent upon the particular dimensions of the catheter and of the device to be held thereby. In the preferred embodiment, the fingers are arranged on the device support region in sets. They may be grouped radially around the device support region or they may be grouped longitudinally along the device support region. In a particular embodiment, the fingers are grouped both in the radial and in the longitudinal direction.

According to another aspect of the present invention, there is provided an introducer system including a catheter element as specified herein, a sheath and a device to be deployed in a patient.

Preferably, the device is a stent, a stent graft, a vena cava filter or an occlusion device.

### DESCRIPTION OF THE DRAWINGS

Embodiments of the present invention are described below, by way only, with reference to the accompanying drawings, in which:

FIG. 1 is a side elevational view of an example of known stent delivery device which can be modified to include a catheter element according to the teachings herein;

FIGS. 2 and 3 show the stent delivery device of FIG. 1 during deployment of a stent;

FIG. 4 is a side elevational view of the distal end of an embodiment of a catheter;

FIG. 5 is a view of the catheter of FIG. 4 from its distal end;

FIG. 6 is a side elevational view of another embodiment of a catheter having hooked fingers;

FIG. 7 is a side elevational view of an embodiment of a catheter having fingers at an angle of between 20° to 80° relative to the longitudinal direction of the catheter;

FIG. 8 is a side elevational view of another embodiment of a catheter having a plurality of sets of fingers arranged in the longitudinal direction of the catheter;

FIG. 9 is a view of another embodiment of a catheter having a plurality of sets of fingers arranged in the radial direction of the catheter;

FIG. 10 is a side elevational view of another embodiment of a catheter having curved fingers;

FIG. 11 is an elevational view in cross-section of the embodiment of a catheter of FIG. 4 with a stent located thereon; and

FIG. 12 is a perspective view of another embodiment of a catheter.

### DESCRIPTION OF THE PREFERRED EMBODIMENTS

It is to be understood that the Figures are schematic and do not show the various components in their actual scale. In many instances, the Figures show scaled up components to assist the reader.

In this description, when referring to an introducer or deployment assembly, the term distal is used to refer to an end of a component which in use is furthest from the surgeon during the medical procedure, including within a patient. The term proximal is used to refer to an end of a component closest to the surgeon and in practice in or adjacent an external manipulation part of the deployment of treatment apparatus.

On the other hand, when referring to an implant such as a stent or stent graft, the term proximal refers to a location which in use is closest to the patient's heart, in the case of a vascular implant, and the term distal refers to a location furthest from the patient's heart.

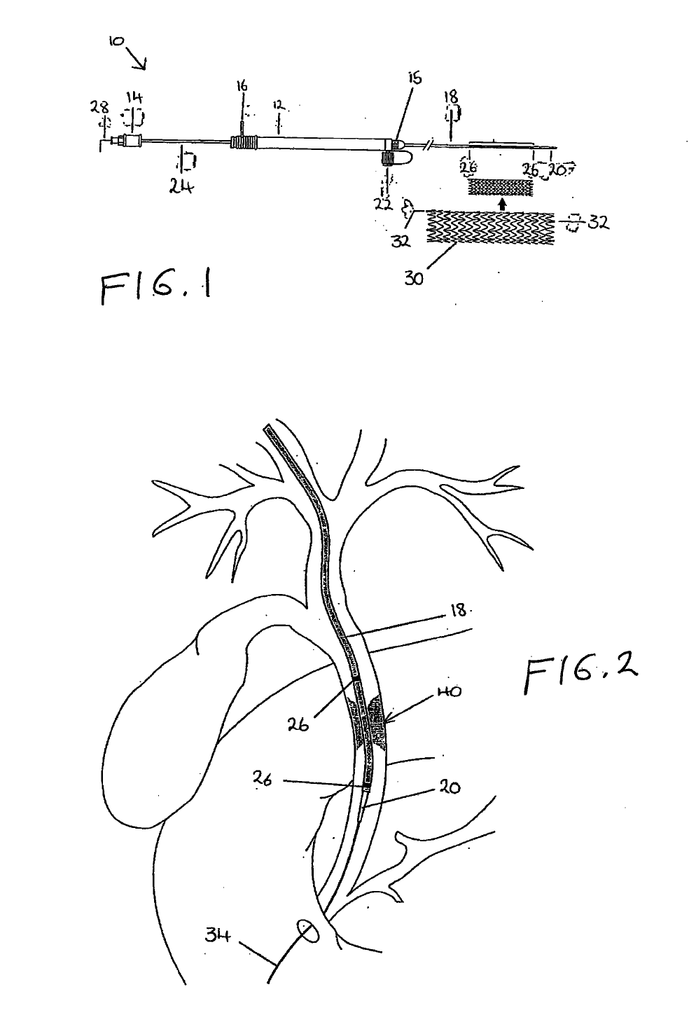

The example of delivery system shown in FIGS. 1 to 3 is the applicant's delivery system for its Zilver™ stent and in particular for its Zilver biliary stent.

The delivery assembly 10 shown in FIG. 1 includes a tubular handle 12, conventionally made of a plastics material, and a hub 14, also made of a plastics material. A safety lock 16 is removably fitted into a portion of the handle 12, for purposes to be described below.

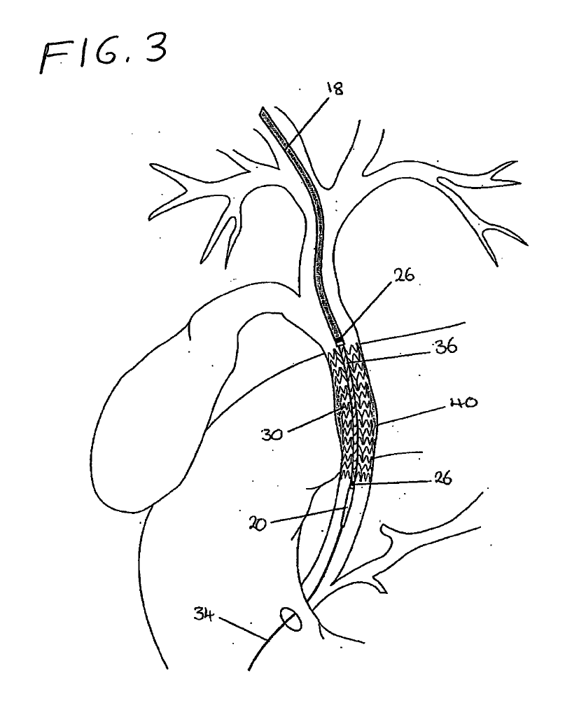

An introducer catheter 18, made of any of the conventional or otherwise suitable catheter materials known in the art, extends from and is attached to the handle 12, in this example by a threaded nut 15. Housed within the introducer catheter 18 is an inner catheter 36 (visible in FIG. 3) which carries stent 30 and which is provided at its distal end with a flexible dilator tip 20. The inner catheter 36 has a bore passing therethrough for the introduction of a guide wire 34, shown in FIGS. 2 and 3.

The handle 12 is provided with a side arm flushing port 22, of conventional form, for flushing the space inside the introducer catheter 18.

The hub 14 is fixed to a metal cannula 24 which is itself attached to the inner catheter 36.

The introducer system 10 is provided with radiopaque markers 26. In this example, the proximal marker 26 is located on the introducer catheter 18, while the distal marker 26 is provided on the inner catheter 36, as will be apparent from FIG. 3.

The hub 14 is provided with an inner support stylet 28 operable to receive and support a guide wire 34, which guide wire 34 passes through the inner stylet 28, the hub 14, the metal cannula 24, the inner catheter 26 and out of distal end of the introducer tip 20.

The distal end of the inner catheter 36, adjacent the dilator tip 20, supports a stent 30, in this example a Zilver™ biliary stent obtainable from the applicant. The introducer catheter 18 overlies and acts as a holding sheath for the stent 30. This stent 30 is provided, in this example, with its own radiopaque markers 32, in a form known in the art.

The safety lock 16 acts to lock the metal cannula in an extended position relative to the handle 12, as shown in FIG. 1, and thus to lock the introducer catheter over the inner catheter 36, until the time of deployment.

Referring now to FIGS. 2 and 3, a stent is deployed, in this case in a biliary tract of a patient, by first introducing a guide wire 34 through an access catheter (not shown) across the distal segment of the target lesion 40 of the biliary tract. Once the guide wire 34 is in place, the introducer catheter 18 is fed over the guide wire 34 until the distal end of the introducer catheter is over the target lesion 40. During this process the introducer catheter is flushed with saline solution through the side arm flushing port 22.

Once the introducer catheter 18 has been located at the deployment site, the stent 30 held by the device 10 is ready to be deployed. This position of the introducer assembly 10 is shown in FIG. 2, with the two fluorescent markers 26 appearing either side of the target lesion site 40.

In order to deploy the stent 30, the safety lock 16 is removed, which allows the handle 12 to be slid over the metal cannula 24. In other words, once the safety lock 16 has been removed, the handle 12 can be pulled back whilst holding the hub 14 steady. This action of pulling back the handle 12 retracts the introducer catheter 18 from the inner catheter 36 with the result that the stent 30 is exposed and allowed to expand gradually as the introducer catheter 18 moves backwards relative to the inner catheter 36. FIG. 3 shows the introducer catheter 18 fully withdrawn and the stent 30 fully deployed at the target lesion 40.

Once the stent 30 has been deployed, the delivery assembly can be withdrawn by pulling the handle 12 and the hub 14 together in a withdrawal direction, that is out of the patient. This procedure is known in the art in particular in connection with deployment of the applicant's Zilver™ stent.

As has been explained above, in some instances, it is possible that friction can develop between the introducer catheter 18 and the stent 30 with the result that the stent 30 can in some instances become deformed as the introducer catheter 18 is withdrawn, typically by compression of the stent.

FIG. 4 shows in side elevation the distal end of an embodiment of catheter 100 for use in the assembly of FIG. 1. The catheter 100 is of a structure substantially similar to conventional inner catheters or cannulae, including those arranged with pushers and/or dilators and other elements used to deploy devices intraluminally and within organs of a patient.

In the embodiment of FIG. 4, the device holding region or element 102 of the catheter 100, that is the region on which the device to be implanted is fitted for deployment, is provided with a plurality of flexible fingers 104 extending outwardly from the holding region 102 of the catheter 100.

The holding region 102 is typically a portion of the catheter 100 designed to hold the device to be implanted and may, for example, have a smaller outer diameter than the remainder of the catheter 100 and may be provided with a shoulder at its proximal end for applying a pushing pressure to the device carried thereby.

The flexible fingers 104 are able to engage with a device being carried on the catheter 100 so as to provide support to the device in the longitudinal direction of the catheter. This is described in more detail below in connection with FIG. 11.

The fingers 104 are preferably substantially uniform throughout their length but it is envisaged that in some embodiments these may have varying flexibilities, for example to become more flexible towards their tips 106. This may be achieved by tapering the thickness or diameter of the fingers 104 towards their tips 106 although it is envisaged that this could also be achieved by use of different materials within each finger.

The fingers 104 may be substantially flat, they may be substantially round or oval in cross-section or may have any other suitable shape.

It is preferred that the array of fingers 102 extends for at least the length of the holding region. In some embodiments, the array of fingers 104 might extend for only a portion of the holding region 102. In fact, in some applications it is not necessary for the fingers 104 to extend over the full length of a device to be carried on the holding element 102.

FIG. 6 shows an embodiment of catheter 200 in which at least some of the fingers 204 have hooked ends 206. These can have the function of hooking over the strut of a stent located on the holding region such as to hold the stent better on the catheter 200. This can reduce the force required to be applied by the sheath to compress the stent, particularly in the case where restraining wires are not used, and thus reduce the friction between the sheath and the stent.

It is preferred that the hooked fingers 204 provide a restraining force which is not sufficient to hold the device in a compressed state on the catheter 200, thus allowing the device to expand normally once the sheath is withdrawn.

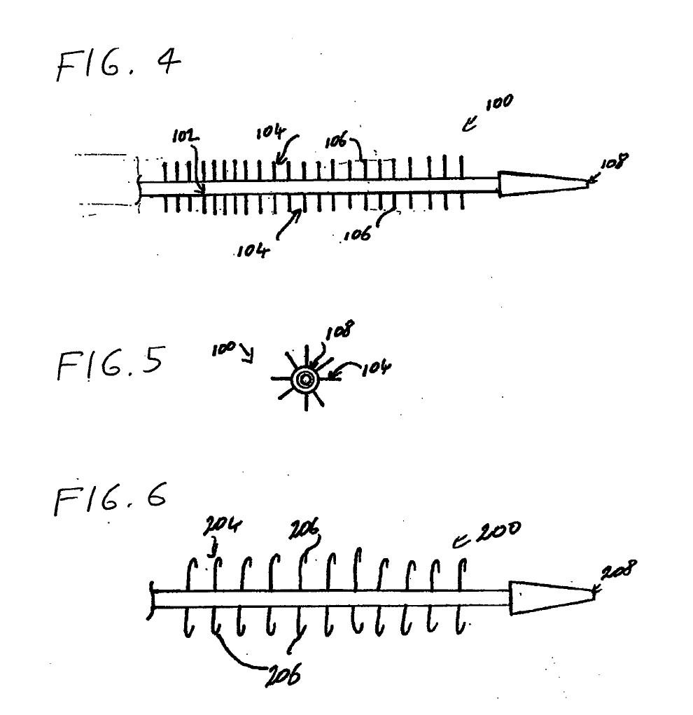

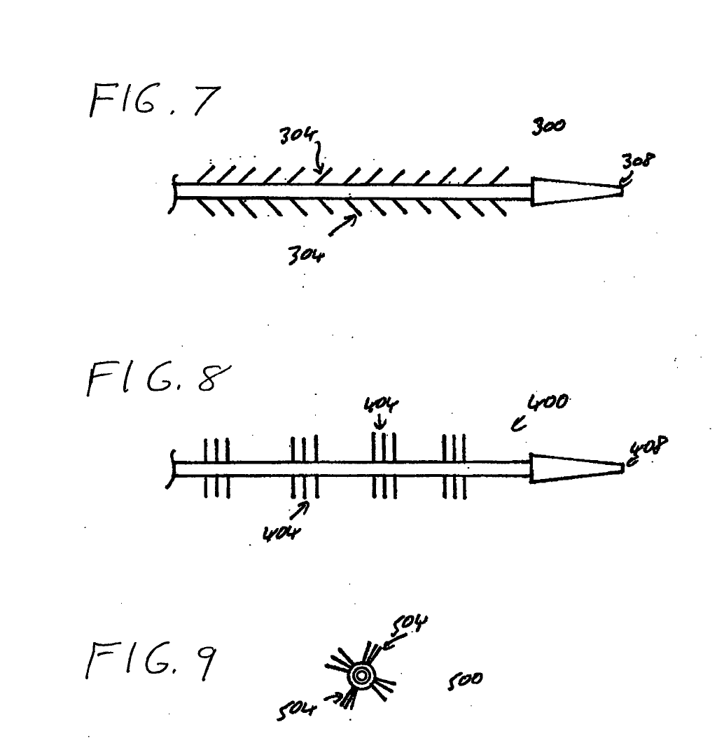

FIGS. 4 and 5 in particular show the fingers 104 extending substantially perpendicularly from longitudinal axis of the holding region 102 of the catheter 100. In some embodiments, some or all of the fingers 102 could extend at another angle. FIG. 7 shows an embodiment of catheter 300 in which the fingers 304 extend outwardly and towards the distal end 308 of the catheter 300. This angle may be, for example, 45° or 60° or any angle between 20° to 80°, more preferably 30° to 60°.

It is envisaged that there could be a variety of different sets of fingers 304, set at different angles to one another.

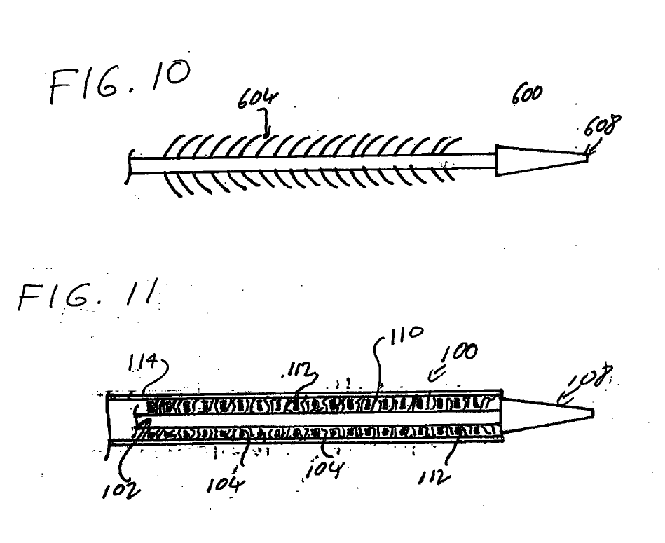

The fingers 104 are shown in FIGS. 4 and 5 to be substantially evenly spaced along and around the holding region 102. However, other embodiments are envisaged. FIG. 8, for example, depicts an embodiment of catheter 400 having a plurality of sets 404 of fingers which are spaced from one another in the longitudinal direction of the catheter 400. FIG. 9 shows an end view of an embodiment of catheter 500 having a plurality of sets 504 of fingers which are spaced from one another in the radial direction of the catheter 500. It is also envisaged that the fingers may be grouped both in the radial and in the longitudinal direction.

The fingers 104 may be substantially straight, as shown in FIGS. 4 and 5, but they could equally be curved as shown in FIG. 10, preferably in a direction towards the distal end 608 of the catheter 600. In some embodiments, the catheter 600 could be provided with a mixture or straight curved fingers. Equally, in some embodiments there could be fingers which curve away from the distal end 608.

In another embodiment, the fingers may be arranged in one or more helixes around the elongate element 102.

The fingers may be formed from the same material as the elongate element 102, and in practice as the catheter 100. This allows the fingers to be moulded with the catheter.

It is also envisaged that the fingers could be are formed from a material different from the elongate element and the catheter, such as of a fibrous material such as metal, metal alloy, Nitinol, nylon. In this case, the fingers could be embedded, welded or adhered onto the holding portion of the catheter.

The length of the fingers will be dependent upon the particular dimensions of the catheter and of the device to be held thereby. In some embodiments, the fingers will be of a length to touch the inner surface of the outer sheath. They may also be longer than this.

The fingers need not be the same length as one another. They could, for example, decrease in length from one end of the element 102 to the other or could decrease in length towards or away from its centre.

FIGS. 4 and 5 show a plurality of rows of fingers 104 extending along the elongate element 102, in particular eight rows. In other embodiments a different number of rows may be provided, including just two located diametrically opposite one another.

FIG. 11 shows a cross-sectional view of the embodiment of catheter 100 of FIGS. 4 and 5 with a stent 110 thereon. The stent 110 is formed of a plurality of interconnected struts 112 which sit between adjacent fingers 104 of the catheter 100. The fingers 104 have the effect of providing resistance to longitudinal movement of the stent 110 when the sheath 114 is retracted during the deployment operation and in many embodiments also resistance against twisting of the stent 110 in a radial direction, caused for example by twisting or bending of the catheter 100 or the sheath 114 during withdrawal of the sheath 114.

Given the flexible nature of the fingers 104, as a stent is being compressed onto the catheter 100 during the assembly process, the fingers 104 will tend to deflect out of their way, in a manner shown in FIG. 11.

Furthermore, flexible nature of the fingers prevents or substantially eliminates the risk of damage to the stent or to the patient during withdrawal of the catheter once the stent has been deployed. In particular, even with fingers which are substantially evenly flexible throughout their length, the tips 106 of the fingers will be able to deflect more than their bases, with the result that if they come into contact with the deployed device or the walls of the patient's vasculature or organ, they will brush against these without causing damage.

Although FIG. 11 shows a stent 110 carried on the catheter 100, any other device could likewise be carried, including for example a stent graft, a vena cava filter or an occlusion device.

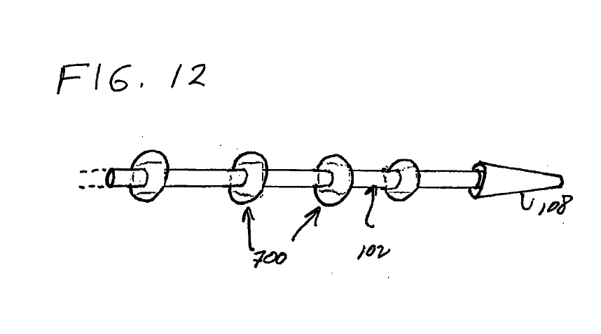

In another embodiment, in place of fingers 104, there may be provided one or more discs on the elongate member 102, as shown in FIG. 12. The discs 700 are made from a flexible material such as silicon, nylon or any other suitable biocompatible material. The discs 700 extend annually around the elongate carrier element 102 at spaced intervals. The discs are sufficiently thin to be able to fold when a stent is compressed onto the elongate carrier 102, such that parts of the discs 700 not directly under a stent strut are not completely compressed and extend into spaces between the stent struts to hold the stent on the elongate carrier 102.

In an alternative embodiment, the discs could instead be in the form of an annular connector for fixing to the elongate carrier, with formed integrally thereon a series of fingers extending annularly from the annular connector so as to fan out from the connector.

Thus, in the described embodiments, the flexible member, that is the fibres, fingers of disks, are able to flex in such a manner as to extend into the interstices of the medical device so as to hold this in position longitudinally on the carrier.

It will be appreciated that the various features of the fingers disclosed herein, including but not limited to the features of curvature, hooked ends, flexibilities and placement in sets may be combined with one another as desired by the skilled person and not restricted to the particular embodiments in which they are described.

The skilled person will also appreciate that there are many methods available in the art for producing such catheter structures with fingers or discs thereon, including for example moulding, adhesion, welding and the like. It is therefore not necessary to describe any such methods in detail herein.

Moreover, although the preferred embodiments have been described in relation to the applicant's Zilver™ stent and delivery system, the teachings herein are applicable to all other catheter or cannula based delivery systems suitable for delivering stents, stent-grafts, filters, occlusion devices and other implants.

1. A catheter element for an introducer, designed to carry a medical device to be implanted in a patient, the medical device comprising a structure with one or more interstices therein;
the catheter element including an elongate device support region on which a medical device can be located and at least one flexible member arranged on at least a portion of said support region, said flexible member extending radially outwardly from said support region and being partially deformable by a medical device carried on the catheter element so as to be at least partially locatable in at least one interstice of the medical device.

2. The catheter element according to claim 1, wherein there is provided a plurality of flexible elements in the form of fingers.

3. The catheter element according to claim 2, wherein the fingers are substantially round, oval or flat, in cross-section or any combination thereof.

4. The catheter element according to claim 2, wherein at least some of the fingers extend substantially perpendicularly to a longitudinal axis of the device.

5. The catheter element according to claim 2, wherein at least some of the fingers extend at an angle to the transverse direction of the device support region.

6. The catheter element according to claim 5, wherein the fingers that extend at an angle comprises an angle of between 20° to 80° in a direction towards or away from a distal end of the catheter.

7. The catheter element according to claim 2, wherein the fingers extend in a radial direction of the device support region.

8. The catheter element according to claim 2, wherein at least some of the fingers are substantially straight.

9. The catheter element according to claim 2, wherein at least some of the fingers are curved.

10. The catheter element according to claim 2, wherein at least some of the fingers have hooked ends.

11. The catheter element according to claim 2, wherein the fingers are formed from the same material as the elongate element.

12. The catheter element according to claim 2, wherein the fingers are formed from a material different from the elongate element and the catheter.

13. The catheter element according to claim 2, wherein the fingers are arranged on the elongate element in sets.

14. The catheter element according to claim 13, wherein the fingers are grouped in sets radially around and/or longitudinally along the device support region.

15. The catheter element according to any preceding claim 1, wherein the device support region is a portion of the catheter designed to hold the device to be implanted.

16. An introducer system including a catheter element according to claim 1, a sheath and a medical device to be deployed in a patient.

17. The introducer system according to claim 16, wherein the medical device is a stent, a stent graft, a vena cava filter or an occlusion device.

18. An introducer system including a catheter element designed to carry a medical device to be implanted in a patient, which medical device includes a structure with one or more interstices therein;
the catheter element including an elongate device support region on which a device can be located and a plurality of flexible members in the form of fingers arranged on at least a portion of said support region, said flexible members extending radially outwardly from said support region and being partially deformable by a said medical device carried on the catheter element so as to be at least partially locatable in the interstice or interstices of the medical device.

19. The introducer system according to claim 18, wherein at least some of the fingers extend at an angle that is transverse to the device support region

20. The introducer system according to claim 18, wherein at least some of the fingers are curved.

21. The introducer system according to claim 18, wherein at least some of the fingers have hooked ends.
